(19) United States
(12) Patent Application Publication
(10) Pub. No.: US 2022/0040287 A1
(43) Pub. Date: Feb. 10, 2022

(54) RECOMBINANT AVIAN HERPES VIRUSES CONTAINING MULTIPLE FOREIGN GENES

(71) Applicant: CEVA SANTE ANIMALE, LIBOURNE (FR)

(72) Inventor: Ayumi Fujisawa, Kanagawa (JP)

(21) Appl. No.: 17/414,267

(22) PCT Filed: Dec. 20, 2019
(86) PCT No.: PCT/EP2019/086670

§ 371 Date: Jun. 15, 2021

(30) Foreign Application Priority Data
Dec. 21, 2018 (EP) 18306798.2

(51) Int. Cl. A61K 39/12; C12N 15/86

(52) U.S. Cl. CPC A61K 39/12; A61K 2039/5256; C12N 15/86

(57) ABSTRACT

The present invention relates to recombinant avian herpes viruses containing multiple genes inserted into separate intergenic regions, their manufacture, compositions comprising the same, and the uses thereof.

### FIELD OF THE INVENTION

The present invention relates to recombinant herpes viruses in which at least two recombinant nucleotide sequences have been inserted, and the uses thereof. The invention is particularly suited for producing multivalent viruses or compositions, such as vaccines, that can induce a protective immunity against avian pathogen(s) or disease(s).

### BACKGROUND OF THE INVENTION

Poultry meat and eggs are important food sources, whose consumption increases continually due to the growth of the human population and their great quality-price ratio. In order to ensure poultry health as well as food safety and security, poultry vaccine technology has become a worldwide concern.

Viral vectors expressing pathogen proteins are commonly used as poultry vaccines against targeted pathogens. Vaccines including such viral vectors induce expression of foreign pathogen proteins within infected hosts, which may lead to protective immunity.

Many different classes of viruses have been investigated as candidate vectors for vaccination of avians, such as adenoviruses, AAVs, fowlpox viruses, herpes viruses, and the like.

Three types of herpes viruses have been determined, MDV1, MDV2 and MDV3 (also known as herpes virus of turkey (HVT)). High similarity exists between said viruses (see Kingham et al., Journal of General Virology (2001) 82, 1123-1135) and they all have been used to prepare recombinant viruses in which a foreign gene derived from a pathogen has been integrated, for use as a vaccine in avians, particularly poultry, such as chicken.

Although such vaccine preparations provide efficient results to vaccinate avian species against many diseases, competition and immunosuppression between pathogens can occur when avians are injected with two or more recombinant herpes viruses, each encoding a different antigen.

In order to overcome such interference and also to facilitate vaccination against multiple diseases, various attempts have been made to produce multivalent herpes viruses encoding several antigens.

First studies inserted several genes in a single cloning site in the genome of the herpes virus (see e.g., EP1026246). However, such constructs either did not provide the required level of protective immunity or turned out to be unstable, all or part of the foreign genes being deleted during repeating passages in culture cells.

WO2013/144355 reports stable herpes viruses encoding multiple foreign antigens obtained using combinations of cloning sites located in non-coding regions of the viral genome.

WO2013/057236, WO2013/082327 and WO2013/082317 report another approach in the design of multivalent HVT, by cloning at least one gene within the US2 coding sequence of herpes viruses. According to these applications, US2 would be non-essential for replication of the virus, although such property is not verified.

Considering the number of pathogens and species, there is a need in the art for further recombinant multivalent herpes viruses which can stably express multiple genes in vivo and are suitable for vaccination of avians, particularly poultry.

### SUMMARY OF THE INVENTION

The present invention provides recombinant avian herpes viruses containing multiple recombinant nucleotide sequences in at least two separate locations of the viral genome.

More specifically, the invention provides recombinant herpes viruses of turkey (HVT) comprising at least a first and a second recombinant nucleotide sequences, each recombinant nucleotide sequence encoding a polypeptide, wherein the first recombinant nucleotide sequence is inserted into the intergenic region of the viral genome located between HVT053 (UL45) and HVT054 (UL46), and wherein the second recombinant nucleotide sequence is inserted into an intergenic region of the viral genome located between HVT064 (LORF3) and HVT070, particularly between HVT064 (LORF3) and HVT065 (UL55), between HVT065 (UL55) and HVT066 (LORF4), between HVT066 (LORF4) and HVT067 (LORF5), between HVT067 (LORF5) and HVT069 (LORF6), or between HVT069 (LORF6) and HVT070, even more particularly between HVT065 (UL55) and HVT066 (LORF4).

Each recombinant nucleotide may encode the same or a different polypeptide, particularly an antigen, a cytokine, an adjuvant, a hormone, and the like. In a particular embodiment, each recombinant nucleotide encodes an antigen, which may be the same or different (or identical or distinct portions of a same antigen) and may be from a same or from distinct pathogens. The antigen(s) may be selected or derived from e.g., surface proteins, secreted proteins or structural proteins of said pathogen(s), or immunogenic fragments thereof.

The invention also relates to a nucleic acid comprising the genome of a recombinant HVT as defined above, and to a vector (such as a plasmid) containing such a nucleic acid.

The invention further relates to a cell containing a recombinant HVT or a nucleic acid or vector as defined above.

A further object of the invention is a composition comprising a recombinant HVT as defined above and a suitable excipient or diluent.

A further object of the invention is a composition comprising a nucleic acid or a cell as defined above and a suitable excipient or diluent.

Another object of the invention resides in a vaccine which comprises an effective immunizing amount of a recombinant HVT, nucleic acid and/or cell, as defined above.

A further object of the invention resides in a recombinant HVT, nucleic acid or cell as defined above, for use for immunizing an avian, such as poultry, against a pathogen.

A further object of the invention resides in a recombinant HVT, nucleic acid or cell as defined above, for use for protecting an avian, such as poultry, against a disease caused by a pathogen.

A further object of the invention resides in a vaccine as defined above, for use for vaccinating an avian, such as poultry, against one or more pathogen(s).

A further object of the invention resides in a method for vaccinating an avian comprising administering to the avian a composition or vaccine or virus as defined above.

A further object of the invention resides in a method for inducing an immune response to an antigen in an avian comprising administering to the avian a composition or vaccine or virus as defined above.

The invention also provides a vaccination kit for immunizing an avian which comprises the following components:

a. an effective amount of a composition or vaccine as defined above, and

b. means for administering said composition or vaccine to said avian.

The invention may be used in any avian, for vaccination against any avian pathogen. It is particularly suited to vaccinate poultry, such as chicken.

### BRIEF DESCRIPTION OF THE DRAWINGS

FIG. 1 illustrates the schematic diagram of the HVT genome (1A). The location of the Unique Long (UL) 45(HVT053) and UL 46(HVT054) and the location of the Unique Long HVT064-070 are marked. Recombinant nucleotide sequences can be inserted at PCR-generated SfiI sites between HVT053 (UL45) and HVT054 (UL46), and between HVT064 and 065, and/or HVT065 and 066, and/or HVT066 and 067, and/or HVT 067 and 069, and/or HVT 069 and 070. HVT constructs integrating different clusters of nucleotide sequences and promoters, according to particular embodiments of the invention are also represented (1B).

FIG. 2A shows immunofluorescence staining of CEFs infected with double recombinant HVTs according to embodiments of the invention (FW241 and FW242) co-expressing NDV-F and ILTV-gB (rHVT/ND/ILT infected cells). Protein F expression was detected by anti-F Mab (77-2) and Alexa Flour 488. Protein gB expression was detected by anti-gBN4 rabbit serum and Alexa Flour 546. The results show that both cells infected with FW241 or FW242 express both the inserted NDV-F protein and the inserted ILTV-gB protein. They were observed by fluorescence microscope at magnification by 40 times.

FIG. 2B shows immunofluorescence staining of CEFs infected with double recombinant HVTs according to embodiments of the invention (FW259) co-expressing IBDV VP2 and ILTV-gB (rHVT/IBD/ILT infected cells). Protein VP2 expression was detected by anti-VP2 Mab (R63) and Alexa Flour 488. Protein gB expression was detected by anti-gBN4 rabbit serum and Alexa Flour 546. The results show that the cells infected with FW259 express both the inserted IBDV-VP2 protein and the inserted ILTV-gB protein. They were observed by fluorescence microscope at magnification by 100 times.

FIGS. 3A and 3B are western blotting analysis showing the expression of F protein and/or gB protein in CEF infected with rHVTs of the invention. As shown in FIG. 3 A, a protein band of 60 kilodaltons (kDa) was observed only in the lane with rHVT/ND/ILT infected cells, which was the expected size of the F protein ( [symbol] ). There was no band in the lane of rHVT/45-46BacVP2 (FW181). As shown in FIG. 3B, gB protein was observed at 54-kilodaltons (kd) in the lanes of each rHVT/ND/ILT ( [symbol] ). On the contrary, there was no band in the lane of rHVT/45-46 PecF (FW168). Double rHVTs of the invention expressed both NDV-F and ILTV-gB.

FIGS. 4A and 4B shows results of a PCR analysis for stability check of recombinant HVT FW242 in successive passages, indicating that after 20 passages F gene and gB gene were expressed stably in the rHVT FW242 of the invention.

FIGS. 4C and 4D shows results of a PCR analysis for genome structure check of purified FW243, and stability check of FW243 in successive passages. They indicated that FW243, double recombinant HVT/ND/ILT of the invention had the expected genomic structure and after 20 passages F gene and gB gene were expressed stably in the rHVT FW243 of the invention.

FIGS. 5A and 5B show results of a western blotting analysis for stability check of recombinant HVT FW244 in successive passages, indicating that after 20 passages F protein and gB protein were expressed stably in CEF infected with the rHVT FW244 of the invention.

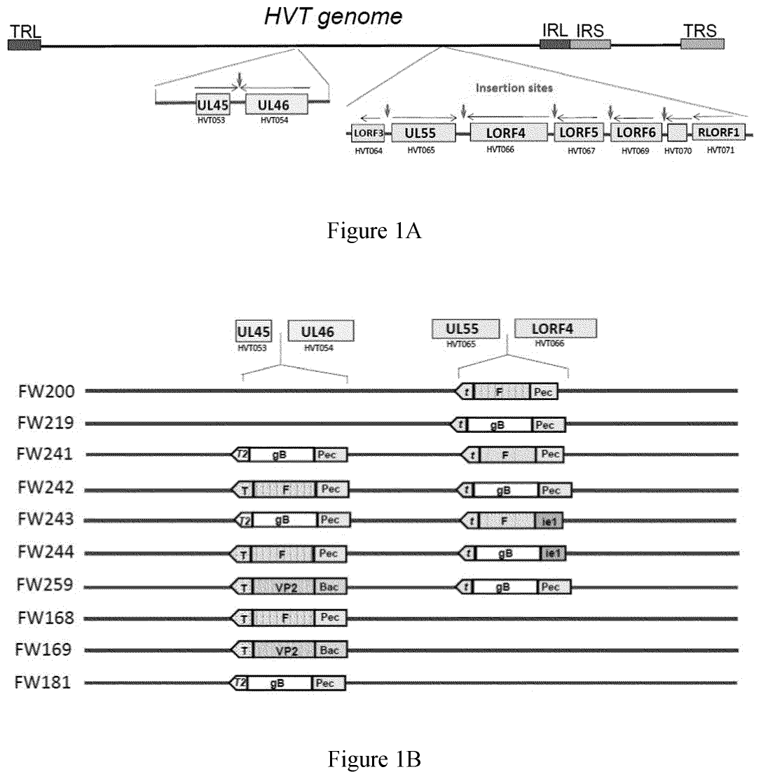

### DETAILED DESCRIPTION OF THE INVENTION

The present invention generally relates to recombinant avian herpes viruses containing multiple recombinant nucleotide sequences, their manufacture, compositions comprising the same, and the uses thereof.

### Definitions

The present disclosure will be best understood by reference to the following definitions:

The term “recombinant”, in relation to a herpes virus, refers to a herpes virus the genome of which has been modified by insertion of at least one nucleotide sequence (e.g., DNA, such as a gene) which is not found naturally in the genome of the herpes virus, or which is found naturally in said genome but in a different form or at a different position. It will be understood that the recombinant herpes virus can be manufactured by a variety of methods such as recombinant DNA technology as described therein and, once made, can be reproduced without further use of recombinant DNA technology. The structure of the “recombinant herpes virus” is therefore described in terms of DNA insertion.

In the present description, the terms “nucleic acid”, “nucleic sequence,” and “nucleotide sequence” are used interchangeably and refer to a nucleic acid molecule having a determined sequence, which may be deoxyribonucleotides and/or ribonucleotides. The nucleotide sequence may be first prepared by e.g., recombinant, enzymatic and/or chemical techniques, and subsequently replicated in a host cell or an in vitro system. A nucleotide sequence preferentially comprises an open reading frame encoding a molecule (e.g. a peptide or protein). The nucleotide sequence may contain additional sequences such as a promoter, a transcription terminator, a signal peptide, an IRES, etc. Preferably, an open reading frame in a recombinant nucleic acid does not contain an intron.

A “recombinant nucleotide sequence” designates a sequence which is not found naturally in the genome of a herpes virus, or which is found naturally in said genome but in a different form or at a different position. Typical “recombinant nucleotide sequences” are genes preferably encoding molecules which are not produced naturally by an avian herpes virus, such as molecules from a different virus or from a cell. A “gene” in the context of such “recombinant nucleotide sequence” designates any nucleic acid molecule containing an open reading frame, such as a nucleic acid consisting or consisting essentially of an open reading frame. The gene may further contain regulatory elements, such as a promoter or terminator, for instance.

In the present description, the terms “polypeptide”, “peptide,” and “protein” are used interchangeably and refer to any molecule comprising a polymer of at least 2 consecutive amino acids.

The term “intergenic region” is well known in the art and refers to any region of a viral genome which is located between two specified viral ORFs. The intergenic region between UL45 (HVT053) and UL46 (HVT054) thus designates the region starting immediately 3′ of the STOP codon of UL45 and ending immediately 5′ of the STOP codon of UL46 (since both ORFs are in opposite orientation). The intergenic region between HVT064 and HVT065 designates the region starting immediately 3′ of the START codon of HVT064 and ending immediately 5′ of the START codon of HVT065 (since both ORFs are in reverse orientation). Similarly, the intergenic region between HVT065 and HVT066 designates the region starting immediately 3′ of the STOP codon of HVT065 and ending immediately 5′ of the STOP codon of HVT066 (since both ORFs are in opposite orientation). The intergenic region may include regulatory regions such as terminators or promoters.

The term “avian species” is intended to encompass all kinds of avians such as birds of the class of Ayes, i.e., vertebrate animals which are feathered, winged, bipedal, endothermic and egg-laying. In the context of the invention, avians or avian species refer more particularly to birds with economical and/or agronomical interests, such as poultry, (such as chickens and turkeys), waterfowl poultry (such as ducks and geese) and ornamental birds (such as swans and psittacines).

The term “vaccine” as used herein designates an agent which may be used to cause, stimulate or amplify an immune response in an organism.

The term “multivalent”, as used herein in relation to a recombinant herpes virus, a vector, or a vaccine of the invention refers to a recombinant herpes virus or a vaccine which comprises at least two recombinant nucleotide sequences as defined above, said sequences being the same or different, and from a same or a different pathogen.

### Multivalent Recombinant HVT

The invention relates to recombinant HVT containing multiple foreign genes in particular locations. More specifically, the invention shows that stable and effective multivalent recombinant HVT may be obtained when:
at least one recombinant nucleotide sequence is cloned in the intergenic region of the genome located between HVT053 and HVT054, and
at least one recombinant nucleotide sequence is cloned in an intergenic region of the genome located between HVT064 and HVT070.

As shown in the examples, when such combination of sites is used, the recombinant HVT is stable over at least 10, preferably at least 15, more preferably at least 20 passages in CEF cells. Furthermore, using such combination of sites, strong and long-lasting expression of the genes is obtained in vivo, sufficient to procure high protective immunity.

Many potential cloning sites exist in a recombinant herpes virus. In particular, there are nearly 397 ORFs, 99 of which are estimated to encode functional protein products, in a herpes virus genome (see Afonso et al., J. Virology 75(2), 2001, 971). Furthermore, some cloning sites suitable for use in the context of monovalent viruses have shown instability when used in the context of multivalent viruses, especially where two different cloning sites are used in a same virus. The invention shows that the combination of the intergenic region between HVT053 and HVT054 with an intergenic region between HVT064 and HVT070, allows stability and suitable level of expression of both cloned recombinant nucleotide sequences in vitro. Such particular recombinant viruses thus represent novel effective agents for inducing potent protective immunity in vivo.

In a particular embodiment, the invention relates to a recombinant HVT containing multiple recombinant nucleic acids (e.g., foreign genes) wherein:
at least one recombinant nucleic acid is cloned in the intergenic region of the genome located between HVT053 and HVT054, and
at least one recombinant nucleic acid is cloned in the intergenic region of the genome located between HVT064 and HVT065.

In a further particular embodiment, the invention relates to a recombinant HVT containing multiple recombinant nucleic acids (e.g., foreign genes) wherein:
at least one recombinant nucleic acid is cloned in the intergenic region of the genome located between HVT053 and HVT054, and
at least one recombinant nucleic acid is cloned in the intergenic region of the genome located between HVT065 and HVT066.

In a further particular embodiment, the invention relates to a recombinant HVT containing multiple recombinant nucleic acids (e.g., foreign genes) wherein:
at least one recombinant nucleic acid is cloned in the intergenic region of the genome located between HVT053 and HVT054, and
at least one recombinant nucleic acid is cloned in the intergenic region of the genome located between HVT066 and HVT067.

In another particular embodiment, the invention relates to a recombinant HVT containing multiple recombinant nucleic acids (e.g., foreign genes) wherein:
at least one recombinant nucleic acid is cloned in the intergenic region of the genome located between HVT053 and HVT054, and
at least one recombinant nucleic acid is cloned in the intergenic region of the genome located between HVT067 and HVT069. Such intergenic region may to a limited extent overlap with HVT068 ORF, which is however located on the complementary strand. Cloning in such intergenic region may thus alter HVT068 coding sequence.

In a further particular embodiment, the invention relates to a recombinant avian HVT containing multiple recombinant nucleic acids (e.g., foreign genes) wherein:
at least one recombinant nucleic acid is cloned in the intergenic region of the genome located between HVT053 and HVT054, and
at least one recombinant nucleic acid is cloned in the intergenic region of the genome located between HVT069 and HVT070.

Recombinant HVT of the invention may be prepared from any HVT, preferably non-pathogenic HVT. An example of a non-pathogenic strain of HVT (MDV3) suitable for use in the invention is the FC126 strain.

The genomic sequence of the FC126 strain is available in the art (Afonso et al., supra; Kingham et al. supra). The location of the quoted intergenic regions can be easily identified by the skilled person using the teachings of the present application, common knowledge and sequence information available in the literature. For instance, Kingham et al. supra, reports the nucleotide sequence of the FC126 reference strain, as well as the location of most ORFs within said genome.

By reference to a FC126 complete genome (GenBank: AF291866.1), the intergenic region between HVT053 and HVT054 corresponds preferably to nucleotides 95323-95443 of the HVT genome, the intergenic region between HVT064 and HVT065 corresponds to nucleotides 111304-111499 of the HVT genome, the intergenic region between HVT065 and HVT066 corresponds to nucleotides 112010-112207 of the HVT genome, the intergenic region between HVT066 and HVT067 corresponds to nucleotides 112766-113107 of the HVT genome, the intergenic region between HVT067 and HVT069 corresponds to nucleotides 113906-114078 of the HVT genome, and the intergenic region between HVT069 and HVT070 corresponds to nucleotides 116731-116831 of the HVT genome.

In the present application, reference to the complete genome of strain FC126 is a reference to said strain as described in Genbank under accession number AF291866.1 at the filing date of the present application.

The recombinant nucleotide sequences inserted in the genome may be in any orientation.

In a preferred embodiment, a recombinant HVT of the invention comprises:
at least one recombinant nucleotide sequence cloned in the intergenic region of the genome between HVT053 and HVT054, and
at least one recombinant nucleotide sequence cloned in the intergenic region of the genome between HVT065 and HVT066.

In a particular embodiment, the nucleic acid inserted in the intergenic region between HVT053 and HVT054 is in the same orientation as HVT054.

In another particular embodiment, the recombinant nucleotide sequence inserted in the intergenic region between HVT065 and HVT066 is in the same orientation as HVT066.

The recombinant nucleotide sequence or inserted nucleic acid sequence may contain (or be operably linked to) regulatory sequences, such as a promoter and/or a terminator. The promoter used may be either a synthetic or natural, endogenous or heterologous promoter. Any promoter may in principle be used, as long as it can effectively function in the target cells or host. In this regard, the promoter may be eukaryotic, prokaryotic, viral or synthetic promoter, capable of directing gene transcription in avian cells in the context of a multivalent vector. Also, each recombinant nucleotide sequence may contain a promoter, which may be the same or different from each other.

Preferentially, the promoter used for each recombinant nucleotide sequence is selected from a chicken beta-actin (Bac) promoter, a Pec promoter, a Murine Cytomegalovirus (Mcmv) ie1 promoter, a Human Cytomegalovirus (Hcmv) promoter, a Simian virus 40 (SV40) promoter, and a Rous Sarcoma virus (RSV) promoter, or any fragments thereof which retain a promoter activity.

A nucleic acid sequence of a chicken Bac promoter is shown in SEQ ID NO: 1, a sequence of a Pec promoter is shown in SEQ ID NO: 2, and a sequence of a Mcmv ie1 promoter is shown in SEQ ID NO: 3. It should be noted that variants of such sequences encoding functional promoters are known and/or can be designed/tested by the skilled artisan, for use in the instant invention.

In a preferred embodiment, the recombinant nucleotide sequence inserted into the intergenic region located between HVT053 and HVT054 contains a Pec promoter or a Bac promoter. The results obtained by the inventors show such promoters are efficient when positioned in said cloning site, in the context of a multivalent vector of the invention.

In another preferred embodiment, the foreign gene inserted into the intergenic region located between HVT065 and HVT066 contains a Pec or Mcmv ie1 promoter. The results obtained by the inventors show such promoters are particularly efficient when positioned in said cloning site, in the context of a multivalent vector of the invention.

Particularly preferred recombinant HVT of the invention contain (i) a recombinant nucleic acid inserted into the intergenic region between HVT065 and HVT066 under control of a Pec or Mcmv ie1 promoter and (ii) a recombinant nucleic acid inserted into the intergenic region between HVT053 and HVT054 under control of a Pec promoter.

### Recombinant Nucleotide Sequence

The recombinant nucleotide sequences may encode any polypeptide of interest. The recombinant nucleotide sequences may encode polypeptides such as antigens, cytokines, hormones, or adjuvants, for instance.

In a particular embodiment, one or each of said at least 2 recombinant nucleotide sequences encode an antigen from a pathogen or an immunogenic fragment thereof.

They may be derived or obtained from any pathogenic organism capable of causing an infection in an avian species. Examples of pathogens that cause infection in avians include viruses, bacteria, fungi, and protozoa.

Antigens may be any immunogenic peptides or proteins of a pathogen, such as a peptide or protein selected from or derived from surface proteins, secreted proteins or structural proteins of said pathogen, or fragments thereof.

Preferred recombinant nucleotide sequences for use in the present invention encode an antigen from avian influenza virus, avian paramyxovirus type 1 (also called Newcastle disease virus (NDV)), avian metapneumovirus, Marek's disease virus, Gumboro disease virus (also called infectious bursal disease virus (IBDV)), Infectious laryngotracheitis virus (ILVT), Infectious bronchitis virus (IBV), Escherichia coli, Salmonella, Pasteurella multocida, Riemerella anatipestifer, Ornithobacterium rhinotracheale, Mycoplasma gallisepticum, Mycoplasma synoviae, Mycoplasmas microorganisms infecting avian species, and/or coccidian.

Preferentially, at least one of the recombinant nucleotide sequences inserted into the viral genome encode an antigen selected from a F protein of NDV, a VP2 protein of IBDV, a gB protein of ILTV, a 40K protein of Mycoplasma gallisepticum, and a surface protein hemagglutinin (HA) of the avian influenza virus, or immunogenic fragments thereof. Preferentially, both recombinant nucleotide sequences encode such an antigen, which may be the same or not.

Immunogenic fragments of an antigen designate any fragment which can elicit an immune response against said antigen in vivo, preferably any fragment which contains an epitope. Immunogenic fragments generally contain from 5 to 50 consecutive amino acid residues of an antigen, such as from 5 to 40, or from 10 to 40.

Various combinations of antigenic peptides may be considered.

In an embodiment, the recombinant HVT of the invention contain a recombinant nucleotide sequence encoding a F protein of NDV or an immunogenic fragment thereof and a nucleotide sequence encoding a VP2 protein of IBDV or an immunogenic fragment thereof.

In another embodiment, the recombinant HVT of the invention contain a nucleotide sequence encoding a VP2 protein of IBDV or an immunogenic fragment thereof and a nucleotide sequence encoding a gB protein of ILTV or an immunogenic fragment thereof.

In a preferred embodiment, the recombinant HVT of the invention contain a nucleotide sequence encoding a F protein of NDV or an immunogenic fragment thereof and a nucleotide sequence encoding a gB protein of ILTV or an immunogenic fragment thereof.

In another variant, a recombinant HVT of the invention expresses two or more antigens from a same pathogen. Said antigens may be the same or different.

In a further variant, at least one of the recombinant nucleotide sequences encode an active molecule such as a cytokine or immunomodulator, an adjuvant, a hormone, an antiparasitic agent, an antibacterial agent, and the like and the other encodes, for instance, an antigen.

According to a further embodiment, three or more recombinant nucleotide sequences are inserted into the viral genome.

### Method of Construction

The recombinant HVT of the invention may be prepared using techniques known per se in the art, such as recombinant technology, homologous recombination, site-specific insertion, mutagenesis, and the like.

Gene cloning and plasmid construction are well known to one person of ordinary skill in the art and may be essentially performed by standard molecular biology techniques (Molecular Cloning: A Laboratory Manual. 4th Edition, Cold Spring Harbor Laboratory Press, Woodbury, N.Y. 2012).

Herpes viruses may be propagated in any suitable host cell and media. The host and the conditions for propagating herpes viruses include, for instance, cells derived from chicken such as CEF (chick embryo fibroblast), chicken kidney cells, and the like. Such cells may be cultured in a culture medium such as Eagle's MEM, Leibowitz-L-15/McCoy 5A (1:1 mixture) culture medium at about 37° C. for 3 to 4 days.

Genomic DNA may be extracted from virus-infected cells according to any conventional method. In particular, after proteins are denatured in a lysis buffer and removed, DNA may be extracted with phenol and ethanol.

Typically, recombinant viruses may be prepared by homologous recombination between a viral genome and a construct (e.g., a plasmid) comprising the recombinant nucleotide sequence or nucleic acid to be inserted, flanked by nucleotides from the insertion site allowing recombination. Briefly, a sequence containing a targeted intergenic region is first cloned into a plasmid, or other suitable vector. Examples of plasmids comprise pBR322, pBR325, pBR327, pBR328, pUC18, pUC19, pUC7, pUC8, and pUC9, examples of phages comprise lambda phage and M13 phage, and example of cosmids comprises pHC79. The cloned region should preferably be of sufficient length so that, upon insertion of the foreign gene, the sequences which flank the foreign gene are of appropriate length so as to allow in vitro homologous recombination with the viral genome. Preferably, each flanking sequence shall have at least approximately 50 nucleotides in length.

In order to insert one or more recombinant nucleotide sequence(s) into the intergenic region, mutation may be carried out at a specific site of the intergenic region to create a cleavage site for a restriction enzyme. A method of carrying out mutation may be a conventional method, and a method commonly used by a person skilled in the art such as in vitro mutagenesis and PCR can be used. Thus, in the PCR method, a mutation such as the deletion, replacement, or addition of 1 to 2 nucleotides in the PCR primer is carried out, and the primer is then used to create a mutation. Alternatively, naturally existing restriction sites may be used. The foreign gene (and promoter) is then inserted into the insertion site of the viral genome in the plasmid.

The resulting plasmid may be introduced into an HV-infected cell or HV genome-transfected cells using any suitable technique (e.g., electroporation, calcium phosphate, a lipofectin-based method or the like). When the amount of the plasmid to be introduced is in the range of 0.1 to 1000 μg, the efficiency of generation of recombinant viruses by recombination between the homologous regions of HV genome and the plasmid becomes high in cells. This results in a recombination event between the plasmid and the viral genome, leading to insertion of the recombinant nucleotide sequence into the virus.

The resulting recombinant virus may be selected genotypically or phenotypically using known techniques of selection, e.g., by hybridization, detecting enzyme activity encoded by a gene co-integrated along with the recombinant nucleic acid sequences or detecting the antigenic peptide expressed by the recombinant herpes virus immunologically.

The selected recombinant herpes virus can be cultured on a large scale in cell culture. Once created, the virus may be propagated in suitable cells.

### Preferred Embodiment

The following recombinant HVT are preferred specific embodiments of the invention. As shown in the examples, they allow strong immune response in vivo against antigens encoded by each recombinant nucleotide sequence.

A particularly preferred recombinant HVT of the invention comprises, (i) inserted in the intergenic region between HVT065 and HVT066, a recombinant nucleotide sequence encoding a F protein of NDV, or an immunogenic fragment thereof, preferentially under control of a Mcmv ie1 promoter and, (ii) inserted in the intergenic region between HVT053 and HVT054, a recombinant nucleotide sequence encoding a gB protein of ILTV, or an immunogenic fragment thereof, preferentially under the control of a Pec promoter (FW243).

Another particularly preferred recombinant HVT of the invention is a recombinant avian herpes virus which comprises, (i) inserted in the intergenic region between HVT065 and HVT066, a recombinant nucleotide sequence encoding a gB protein of ILTV or an immunogenic fragment thereof, preferentially under control of a Pec promoter and, (ii) inserted in the intergenic region between HVT053 and HVT054, a recombinant nucleotide sequence encoding a F protein of NDV, or an immunogenic fragment thereof, preferentially under the control of a Pec promoter (FW242).

Another particularly preferred recombinant HVT of the invention is a recombinant avian herpes virus which comprises, (i) inserted in the intergenic region between HVT065 and HVT066, a recombinant nucleotide sequence encoding a gB protein of ILTV, or an immunogenic fragment thereof, preferentially under control of a Mcmv ie1 promoter and, (ii) inserted in the intergenic region between HVT053 and HVT054, a recombinant nucleotide sequence encoding a F protein of NDV, or an immunogenic fragment thereof, preferentially under the control of a Pec promoter (FW244).

Another particularly preferred recombinant HVT of the invention comprises, (i) inserted in the intergenic region between HVT065 and HVT066, a recombinant nucleotide sequence encoding a F protein of NDV, or an immunogenic fragment thereof, preferentially under control of a Pec promoter and, (ii) inserted in the intergenic region between HVT053 and HVT054, a recombinant nucleotide sequence encoding a gB protein of ILTV, or an immunogenic fragment thereof, preferentially under the control of a Pec promoter (FW241).

Another recombinant HVT of the invention comprises, (i) inserted in the intergenic region between HVT065 and HVT066, a recombinant nucleotide sequence encoding a VP2 protein of IBDV or an immunogenic fragment thereof, preferentially under control a Mcmv ie1 promoter or Pec promoter and, (ii) inserted in the intergenic region between HVT053 and HVT054, a recombinant nucleotide sequence encoding a gB protein of ILTV or an immunogenic fragment thereof, preferentially under the control of a Pec promoter.

A further recombinant HVT of the invention comprises, (i) inserted in the intergenic region between HVT065 and HVT066, a recombinant nucleotide sequence encoding a gB protein of ILTV or an immunogenic fragment thereof, preferentially under control a Mcmv ie1 promoter or Pec promoter and, (ii) inserted in the intergenic region between HVT053 and HVT054, a recombinant nucleotide sequence encoding a VP2 protein of IBDV or an immunogenic fragment thereof, preferentially under the control of a Pec promoter (FW259).

The recombinant HVT of the present invention may be propagated in cell cultures. In preferred embodiments, CEF, embryonated eggs, chicken kidney cells, and the like are used as the host cells for the propagation of recombinant HVT. Multivalent recombinant HVT of the present invention may be cultured in a culture medium such as Eagle's MEM, Leibowitz-L-15/McCoy 5A (1:1 mixture) culture medium at about 37° C. for 3 to 4 days. The infected cells thus obtained are suspended in a culture medium containing 10% dimethyl sulfoxide (DMSO) and stored frozen under liquid nitrogen.

Advantageously, the recombinant multivalent HVT of the invention present a high level of stability through passages, which corresponds to a coexpression of the recombinant nucleotide sequences in cells of avian species, preferably CEF cells, even after 10 or more passages, preferably after 15 passages, even more preferably after 20 passages. In the context of the invention a “passage” or “cell passaging” means a culture of cells in suitable conditions for allowing their growth and keeping them alive until they are 90% to 100% confluent. The passaging step consists on transferring a small number of cells of the previous confluent culture into a new culture medium. An aliquot of the previous confluent culture, containing a few cells, may be diluted in a large volume of fresh medium.

The viruses may be collected or purified using conventional techniques. They may be stored in any suitable medium, frozen and/or lyophilized.

### Nucleic Acids and Cells

A further object of the invention relates to any nucleic acid contained in a virus as defined above. The nucleic acids may be single- or double-stranded, DNA or RNA, or variants thereof.

The invention also relates to a vector (e.g., plasmid, cosmid, artificial chromosome, etc.) containing a nucleic acid of the invention.

The invention also relates to a cell containing a recombinant HVT, nucleic acid or vector of the invention. The cells are typically eukaryotic cells, such as avian cells, or prokaryotic cells (if the vector is suitable for replication or maintenance in such cell type).

### Vaccine Compositions

The invention also relates to compositions, such as vaccines, which comprise a multivalent recombinant HVT of the invention, a nucleic acid of the invention, or a cell of the invention.

Vaccines of the invention typically comprise an immunologically effective amount of a recombinant HVT as described above, in a pharmaceutically acceptable vehicle.

The compositions and vaccines according to the present invention typically comprise a suitable solvent or diluent or excipient, such as for example an aqueous buffer or a phosphate buffer. The compositions may also comprise additives, such as proteins or peptides derived from animals (e.g., hormones, cytokines, co-stimulatory factors), nucleic acids derived from viruses and other sources (e.g., double stranded RNA, CpG), and the like which are administered with the vaccine in an amount sufficient to enhance the immune response. In addition, any number of combinations of the aforementioned substances may provide an immunopotentiation effect, and therefore, can form an immunopotentiator of the present invention.

The vaccines of the present invention may further be formulated with one or more further additives to maintain isotonicity, physiological pH and stability, for example, a buffer such as physiological saline (0.85%), phosphate-buffered saline (PBS), citrate buffers, Tris(hydroxymethyl aminomethane (TRIS), Tris-buffered saline and the like, or an antibiotic, for example, neomycin or streptomycin, etc.

The route of administration can be any route including oral, ocular (e.g., by eyedrop), oculo-nasal administration using aerosol, intranasal, Cloacal in feed, in water, or by spray, in ovo, topically, or by injection (e.g., intravenous, subcutaneous, intramuscular, intraorbital, intraocular, intradermal, and/or intraperitoneal) vaccination. The skilled person will easily adapt the formulation of the vaccine composition for each type of route of administration.

Each vaccine dose may contain a suitable dose sufficient to elicit a protective immune response in avian species. Optimization of such dose is well known in the art. The amount of antigen per dose may be determined by known methods using antigen/anti-body reactions, for example by the ELISA method.

The vaccines of the invention can be administered as single doses or in repeated doses, depending on the vaccination protocol.

The vaccines of the present invention are further advantageous in that they confer to bird species up to 80% protection against the targeted avian pathogens after 3 weeks of vaccination.

The present invention further relates to the use of the vaccine as described above for immunizing avian species, such as poultry, against a pathogen.

The present invention further relates to a method of immunizing avian species by administering an immunologically effective amount of the vaccine according to the invention. The vaccine may be advantageously administered intradermally, subcutaneously, intramuscularly, orally, in ovo, by mucosal administration or via oculo-nasal administration.

The present invention further relates to vaccination kits for immunizing avian species which comprises an effective amount of the multivalent vaccine as described above and a means for administering said components to said species. For example, such kit comprises an injection device filled with the multivalent vaccine according to the invention and instructions for intradermic, subcutaneous, intramuscular, or in ovo injection. Alternatively, the kit comprises a spray/aerosol or eye drop device filled with the multivalent vaccine according to the invention and instructions for oculo-nasal administration, oral or mucosal administration.

Further aspects and advantages of the present application will now be disclosed in the following examples, which are illustrative of the invention.

### EXAMPLES

In the experimental section, several recombinant HVT (monovalent or multivalent according to the invention) have been generated, designated as follows (HVT/first insertion site-first foreign gene/second insertion site-second foreign gene):

### Monovalent:

### FW200: HVT/65-66 Pec F

### FW219: HVT/65-66 Pec gB

### FW168: HVT/45-46 Pec F

### FW169: HVT/45-46 Bac VP2

### FW181: HVT/45-46 Pec gB

### Multivalent:

### FW241: HVT/65-66 Pec F/45-46 Pec gB

FW242: HVT/65-66 Pec gB/45-46 Pec F

### FW243: HVT/65-66 Mcmv ie1 F/45-46 Pec gB

FW244: HVT/65-66 Mcmv ie1 gB/45-46 Pec F
FW259: HVT/65-66 Pec gB/45-46 Bac VP2

### Example 1: Construction of Homology Vectors

The plasmid construction was essentially performed by the standard molecular biology techniques (Molecular Cloning: A Laboratory Manual. 4th Edition, Cold Spring Harbor Laboratory, Cold Spring Harbor, N.Y. 2012). DNA restriction fragments were electrophoresed on agarose gels and purified with Plasmid plus Midi Kit (QIAGEN, Cat #12945).

HVT DNA was prepared from CEF cells infected with the HVT FC126 strain according to the method of Lee et al. (J. Gen. Virol., 51: 245-253, 1980). HVT064-071 were amplified by PCR using the obtained HVT DNA as a template. Two primers were used, one was SEQ ID NO:4 (5′-GGATGTCCAATTCGCACATG-3′) and the other was SEQ ID NO:5 (5-GCTACAGTTACGGGATTCATAGG-3′). Each primer was designed on the information of GenBank AF291866.1.

Construction of pHVT64-65

Using the HVT064-071 as a template, PCR was performed with two pairs of primers. A DNA fragment having a SfiI site between two ORFs, HVT064 (LORF3) and HVT065 (UL55), was prepared by PCR and cloned into pUC18.

The first pair was
SEQ ID NO: 6
(5′-GGGGGAATTCACTACTTTTAATTCTCTTTA-3′)
and
SEQ ID NO: 7
(5′-GGGGGCCAATAAGGCCGCTAGCGGCCGCCTAACACCCCC
GAATATTAGTC-3′).
The second pair was
SEQ ID NO: 8
(5′-GGGGCGGCCGCTAGCGGCCTTATT
GGCCTCAC GTGTAGCCCATTGTGTGCATATAAC-3′)
and
SEQ ID NO: 9
(5′-GGGAAGCTTAG ATCTGAAATAACGCAGTTG-3′).

The first resulting fragment was digested with EcoRI and NheI. The second resulting fragment was digested with NheI and HindIII. These cleaved fragments were integrated into pUC18 cleaved with EcoRI and HindIII, resulting the plasmid pHVT 64-65.

Construction of pHVT65-66

Using the HVT064-071 as a template, PCR was performed with two pairs of primers. A DNA fragment having a SfiI site between two ORFs, HVT065 (UL55) and HVT066 (LORF4), was prepared by PCR and cloned into pUC18.

The first pair was
SEQ ID NO: 10
(5′-GGGGAATTCGCCAGATATCCAAAGTACAGC-3′)
and
SEQ ID NO: 11
(5′-GGGGGCCAATAAGGCCGCTAGCGGCCGCCAATTATTT
TATTTAATAACATAT-3′).
The second pair was
SEQ ID NO: 12
(5′-GGGGCGGCCGCTAGCGGCCTTATTGGC
CACCAGTGAACAATTT GTTTAATGTTA-3′)
and
SEQ ID NO: 13
(5′-GGGAAGCT
TGGGTCTGTCCTAGCGATATAA-3′).

The first resulting fragment was digested with EcoRI and NheI. The second resulting fragment was digested with NheI and HindIII. These cleaved fragments were integrated into pUC18 cleaved with EcoRI and HindIII, resulting the plasmid pHVT 65-66.

Construction of pHVT66-67

Using the HVT064-071 as a template, PCR was performed with two pairs of primers. A DNA fragment having a SfiI site between two ORFs, HVT066 (LORF4) and HVT067 (LORF5), was prepared by PCR and cloned into pUC18.

The first pair was
SEQ ID NO: 14
(5′-GGGGGAATTCTCCAGATTGTTGGATATCTG-3′)
and
SEQ ID NO: 15
(5′-GGGGGCCAATAAGGCCGCTAGCGGCCGCCTTATTG
ATTTATAAAAACATACATGCAGTG-3′).
The second pair was
SEQ ID NO: 16
(5′-GGGGCGGCCGCTAGCGGCCTTATTG
GCCAGTACATAATTTATTACGTATCATTTCCG-3′)
and
SEQ ID NO: 17
(5′-GGGAAG CTTCCTGCAAGACCTCATACGGAA-3′).

The first resulting fragment was digested with EcoRI and NheI. The second resulting fragment was digested with NheI and HindIII. These cleaved fragments were integrated into pUC18 cleaved with EcoRI and HindIII, resulting the plasmid pHVT 66-67.

Construction of pHVT67-69

Using the HVT064-071 as a template, PCR was performed with two pairs of primers. A DNA fragment having a SfiI site between two ORFs, HVT067 (LORF5) and HVT069 (LORF6), was prepared by PCR and cloned into pUC18.

The first pair was
SEQ ID NO: 18
(5′-GGGGGAATTCATTTCTTCATTGCAACGACG-3′)
and
SEQ ID NO: 19
(5′-GGGGGCCAATAAGGCCGCTAGCGGCCGCATGATC
GTGCTCATTACTGCATCG-3′).
The second pair was
SEQ ID NO: 20
(5′-GGGGCGGCCGCTAGCGGCCTTATTG
GCCGGG CGGGGCGATGACGTTCTATTTGC-3′)
and
SEQ ID NO: 21
(5′-GGGAAGCTTAA TACGCAGATTCTTTTCGG-3′).

The first resulting fragment was digested with EcoRI and NheI. The second resulting fragment was digested with NheI and HindIII. These cleaved fragments were integrated into pUC18 cleaved with EcoRI and HindIII, resulting the plasmid pHVT 67-69.

Construction of pHVT69-70

Using the HVT064-071 as a template, PCR was performed with two pairs of primers. A DNA fragment having a SfiI site between two ORFs, HVT069 (LORF6) and HVT070, was prepared by PCR and cloned into pUC18.

The first pair was
SEQ ID NO: 22
(5′-GGGGGAATTCTAAAGAATCGTACATGAGCG-3′)
and
SEQ ID NO: 23
(5′-GGGGGCCAATAAGGCCGCTAGCGGCCGCCTGAT
GTATAAGATTGCCGAAAAG-3′).
The second pair was
SEQ ID NO: 24
(5′-GGGGCGGCCGCTAGCGGCCTTATTGGCCC
GGGTTGCGTGAATACTGGTCACAG-3′)
and
SEQ ID NO: 25
(5′-GGGAAGCTTACG
ATCTGGCAAAAGGGTCC-3′).

The first resulting fragment was digested with EcoRI and NheI. The second resulting fragment was digested with NheI and HindIII. These cleaved fragments were integrated into pUC18 cleaved with EcoRI and HindIII, resulting the plasmid pHVT 69-70.

Construction of the Homology Vector, pHVT65-66 Pec F

SfiI-cleaved pHVT65-66 was dephosphorylated by using Alkaline Phosphatase Shewanella sp. S1B1 Recombinant (PAP) (Funakoshi #DE110). The fragment was ligated with BglI-cleaved p45/46Pec F (WO2003 001066), resulting in the plasmid, pHVT65-66 Pec F. The synthetized short polyA signal (SPA: SEQ ID NO:26 CTGCAGGCGGCCGCTCTAGA GTCGACAATAAAAGATCTTTATTTTCATTAGATCTGTGTGTTGGTTTTTTGTGTG GCCAATAAGGCC) was integrated into pHVT65-66 PecF cleaved with SalI and SfiI, resulting in the homology plasmid, pHVT65-66 Pec F SPA

Construction of the Homology Vector, pHVT65-66 Pec gB

The homology vector, pHVT65-66 Pec F SPA was cleaved with XbaI and NotI. The 5210-bp fragment was ligated with XbaI/NotI-cleaved pHVT87-88 Pec ILgB del (WO2013 144355) (1506 bp) resulting in the homology plasmid, pHVT65-66 Pec gB SPA.

### Chemical Synthesized Mcmv Ie1 Promoter

Mcmv ie1 promoter (SEQ ID NO: 3) was synthesized on the information of 4191-4731 bp in Gene Bank L06816.1 reported by Koszinowski, U. H. Synthesized Mcmv ie1 promoter was designed that BglI-PstI sites were added in front of it and XbaI-NotI sites were added at the end.

### Example 2: Purifying Recombinant HVT in CEF Transfected with Each Transfer Vector

Viral DNA of the HVT wild type, FC126 strain (wt-HVT) was prepared as described by Morgan et al. (Avian Diseases, 34:345-351, 1990). Viral DNAs of FW168 (rHVT/45-46PecF), FW169 (rHVT/45-46BacVP2) and FW181 (rHVT/45-46 PecgBdel) (WO2005 093070) were prepared in the similar method. The first double rHVT pattern was that the CEF cells were transfected with the prepared wt-HVT DNA and pHVT65-66 Pec F (ex. FW200). The second pattern was that the CEF cells were transfected with the prepared FW168 DNA and pHVT65-66 Pec gB (ex. FW242). The third pattern was that the CEF cells were transfected with the prepared FW181 DNA and pHVT65-66 Pec F (ex. FW241). The fourth pattern was that the CEF were transfected with the prepared FW169 DNA and pHVT65-66 Pec gB (ex. FW259). These resulting recombinant viruses were plaque purified by staining plaques with the anti-ILTV gB antibody, the anti-NDV-F antibody or anti-IBDV-VP2 antibody.

Briefly, 10⁷ primary CEF cells were suspended in 100 μl of MEF-1 (Lonza LNJVD-1004) and co-transfected with 1 μg of the homology vector, for example, pHVT65-66 PecF and pHVT65-66 Mcmv ie1 gB, and 2 μg of HVT DNA, for instance, FC126, FW181 and FW168 by electroporation. Electroporation was performed on Nucleofector II. Transfected cells were diluted in 20 ml of Leibovitz's L-15 (GIBCO BRL, Cat. #41300-39), McCoy's 5A Medium (GIBCO BRL, Cat. #21500-061) (1:1) and 4% calf serum (named solution LM (+) medium), spread 100 ul per well of 96 well plate.

Incubate at 37° C. in 5% CO₂ until the plaques became visible, the cells were detached from plates by trypsinization, diluted in freshly prepared secondary CEF cells, transferred equally to two 96-well plates and incubated for 3 days to visualize the plaques. One of two plates was then stained with an antibody by which we detected the inserted gene harbored in each transfer vector as the primary antibody. NDV-F was detected by anti-Fc rabbit serum or Mab77-2. ILTV-gB was detected by anti-ILTV gBN4 rabbit serum or Mab#1_B4_7. IBDV-VP2 was detected by anti-VP2 monoclonal antibody R63 (ATCC #: HB-9490). After detecting the well containing the stained recombinant plaques, cells from the corresponding well of the other plate were recovered, diluted in fresh secondary CEF cells and transferred equally to two 96-well plates to complete the first round of purification. The purification procedure was repeated until every obtained plaque was stained positively by each polyclonal antibody or monoclonal antibody. After then, the double rHVT candidate was stained by another antibody. Finally, expression of the proteins by all plaques of the candidate rHVT was confirmed by dual IFA staining. CEFs infected by each rHVT were fixed with cold Acetone-Methanol (2:1), washed with PBS, reacted with antibody mixture (1:1000 diluted anti gBN4 rabbit serum (or Mab#1_B4_1) and anti Fc rabbit serum (Mab77-2) or anti-VP2 mouse Mab R63) at 37 C in 60 minutes. After washing 3 times with PBS, the cells were reacted with fluorescent antibody mixture (1:1000 diluted Alexa Fluor488 anti rabbit and Alexa Fluor546 anti mouse provided by Invitrogen) at 37 C in 60 minutes. After washing 3 times with PBS, they were observed by fluorescence microscope at magnification by 40 or 100 times.

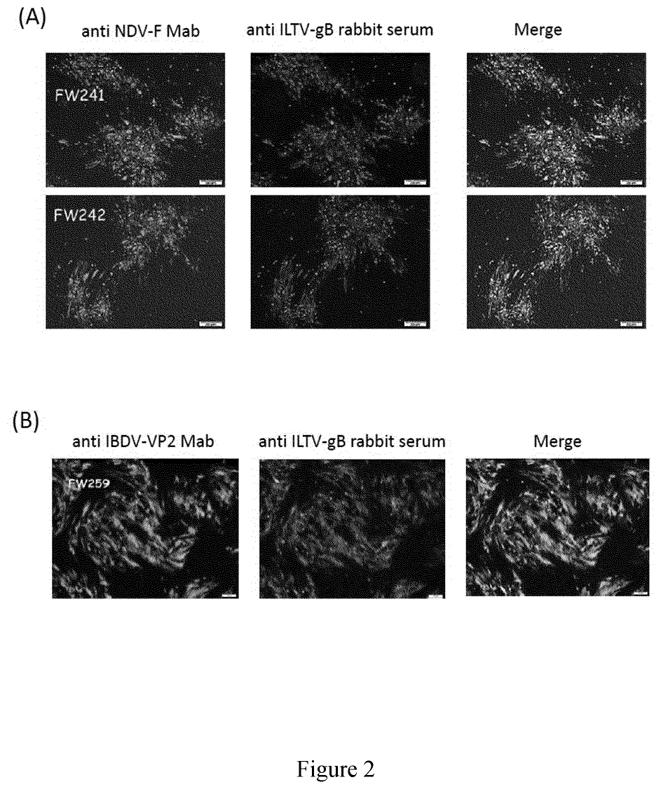

Protein gB expression was detected by anti gBN4 rabbit serum and Alexa Flour 546. Protein F expression was detected by anti-F Mab 77-2 and Alexa Flour 488. Protein VP2 expression was detected by anti-VP2 Mab (R63) and Alexa Flour 546. When all plaques expressed both F and gB (both gB and VP2), we concluded purification was completed. FIGS. 2A and B show some examples of dual IFA.

The purified recombinant HVT was designated rHVT/ND/ILT or rHVT/ILT/IBDV.

The table 1 below shows the expression of the gB and protein F obtained from the different rHVT/ND/ILT.

Strain FW168 (HVT/45-46 PecF) and FW200 (HVT/65-66 PecF) correspond to a monovalent recombinant herpes virus used as control for protein F expression, and FW181 (HVT/45-46 Pec gBdel) and FW219 (HVT/65-66 Pec gBdel) correspond to a monovalent recombinant herpes virus used as control for protein gB expression, and FW169 (HVT/45-46 Bac VP2) corresponds to a monovalent recombinant herpes virus used as control for VP2 expression.

The results show the bivalent constructs of the invention express both antigens.

TABLE 1
Expression of the inserted NDV-F and ILTV gB gene by
rHVT/ND/ILT (Detection of fluorescence)

| | Primary antibody | | |
|---|---|---|---|
| Virus | anti-F Mab | anti-gB antiserum rabbit | PBS |
| FW241 | + | + | − |
| FW242 | + | + | − |
| FW243 | + | + | − |
| FW244 | + | + | − |
| FW200 | + | − | − |
| FW219 | − | + | − |
| FW168 | + | − | − |
| FW181 | − | + | − |
| FC126 | − | − | − |
| None | − | − | − |

+: detected, −: not detected

### Example 3: Co-Expression of Two Proteins in CEF Infected with Double Recombinant HVTs

2 ml containing 2×10⁵ CEF cells was infected with recombinant HVTs, and incubated at 37° C. in 5% CO₂ for 3 days.

Then the culture was centrifuged at 300 g for 3 minutes, and the precipitated cells were resuspended in 100 ul. Laemmli buffer (100 ul) was added to the cell suspension. The resultant mixture was then boiled for 5 min and 5 ul of them was subjected to 10% SDS-polyacrylamide gel electrophoresis. The electrophoresed proteins were transferred from SDS-GEL to a PVDF membrane (Immobilon-P, Millipore), which was blocked in 1% w/v non-fat milk powder in PBS at room temperature for one hour.

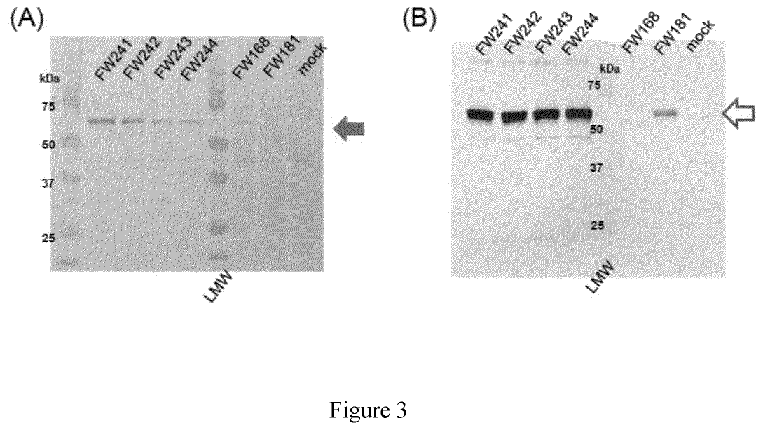

For F detection (FIG. 3A), the treated membrane was then reacted with the anti-Fc rabbit antiserum at 500-fold dilution at room temperature for one hour, washed three times with PBS, and incubated for one hour with the biotinylated anti-rabbit goat antiserum.

For gB detection (FIG. 3B), the treated membrane was then reacted with the anti-gBN4 rabbit serum at 500-fold dilution at room temperature for one hour, washed three times with PBS, and incubated for one hour with the biotinylated anti-rabbit goat antiserum.

After washing three times with PBS, the membrane was incubated for one hour with an avidin-alkaline phosphatase complex, washed three times with PBS and one time with TBS (Tris Buffered Saline), and reacted with BCIP-NBT (a substrate of alkaline phosphatase.) As shown in FIG. 3A, a protein band of 60 kilodaltons (kDa) was observed only in the lane with rHVT/ND/ILT infected cells, which was the expected size of the F protein ( ). There was no band in the lane of rHVT/45-46 Pec gB (FW181).

FIG. 3B shows gB protein was observed at 54-kilodaltons (kd) in the lanes of each rHVT/ND/ILT (⇒). On the contrary, there was no band in the lane of rHVT/45-46 PecF (FW168). The 54-kd is gB truncated protein (469aa; WO2005/093070) which is inserted in rHVT/ND/ILT or rHVT/ILT/IBD.

Double recombinant HVTs according to the invention expressed both NDV-F and ILTV-gB.

### Example 4: Verification of the Genomic Structure

### PCR Analysis

The purified rHVT/ND/ILT was propagated on CEF cells of one 6-well plate to obtain the confluent plaques. Cells were recovered from dishes by scraping, transferred to Falcon tubes and subjected to centrifugation at 300×g for 5 min. One tenth of harvested cells (from one well) were suspended in 0.1 ml of lysis buffer (20 mM Tris-HCl, 100 mM NaCl, 5 mM EDTA, 0.1% SDS), and lysed by vortexing for 1 min. The lysates were incubated at 60° C. for 10 min with 2 μl of Protease K (10 mg/ml). The obtained mixture was treated twice with phenol-chloroform. The aqueous phase (the viral DNA) was used as a template. Primer sets were designed for each insertion site.

For analysis around HVT65/66
65R (SEQ ID NO: 27):
5′-CCACTGCCACTGTGATGATAAG-3′
66F (SEQ ID NO: 28):
5′-GCCTACTATGCACATTGTTACTCCT-3′
For analysis around UL45/46
45-46F-K (SEQ ID NO: 29):
5′-GGGGAAGTCTTCCGGTTAAGGGAC-3′
45-46R (SEQ ID NO: 30):
5′-GGTGCAATTCGTAAGACCGATGGG-3′

PCR was performed using Tks Gflex DNA polymerase (TAKARA BIO Inc. #R060) in accordance with maker's manual.

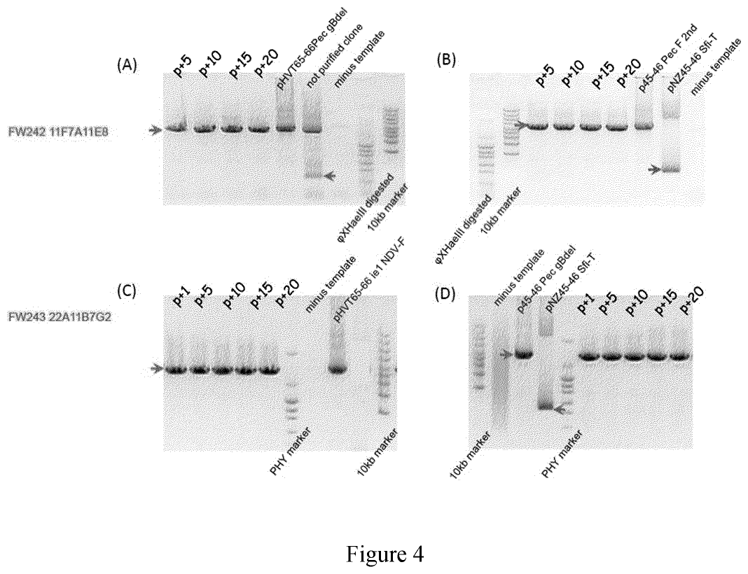

The result of PCR in FIG. 4C shows that a 2550-bp fragment was amplified with the primer set 66F/65R in the DNA from purified FW243 (p+1). The fragment amplified in transfer plasmid pHVT65-66 ie1 NDV-F was the same size. In addition 2281-bp fragment was amplified with another primer set 45-46-K/45-46R in the DNA from purified FW243 (p+1) (FIG. 4D). The same band was detected in p45-46 Pec gBdel. Lower fragment (546 bp) were amplified in pNZ45-46 Sfi-T.

FIG. 4C-D (p+1) indicates that the obtained double recombinants HVT/ND/ILT have the expected genomic structure. The recombinant viruses were confirmed pure because no band corresponding to the parent strains was amplified.

### Example 5: Stability of the Recombinant HVTs in Passage

### PCR Analysis

Each rHVT/ND/ILT was passaged twenty times in CEF cells and subjected to PCR analysis as described in Experiment 4. The results were the same with those obtained in Experiment 4, indicating that the recombinant virus was stable even after 20 passages. The result of PCR in FIG. 4A showed that a 2118-bp fragment was amplified with the primer set 66F/65R in the DNA from FW242 (p+5, +10, +15, +20) and transfer plasmid pHVT65-66 Pec gBdel. In contrast, lower fragment (252 bp) was amplified in a clone which was not purified.

FIG. 4B shows that a 3083-bp fragment was amplified with another primer set 45-46-K/45-46R in FW242 (p+5, +10, +15, +20) and p45-46 Pec F 2^{nd}. Lower band (547 bp) was amplified in pNZ45-46 Sfi-T (U.S. Pat. No. 7,569,365).

FIG. 4C shows that a 2550-bp fragment was amplified in FW243 passaged (p+5, +10, +15, +20). FIG. 4D shows that a 2281-bp fragment was amplified in FW243 passaged (p+5, +10, +15, +20).

PCR analysis with 65R/66F and 45-46-K/45-46R showed F gene or gB gene stably maintained at the insertion site HVT65/66 or UL45/46 respectively in FW242 and FW243.

### Western Blotting Analysis

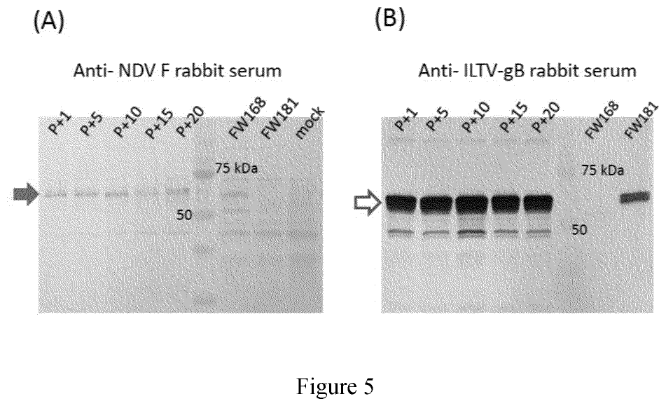

Double recombinant HVTs were passaged serially (up to 20 times) on chicken embryo fibroblasts (CEF). Then Cell lysates were applied to Western blot analysis. In a first panel (FIG. 5A), the blot was reacted with an anti-Fc rabbit serum (1:500). In a second panel (FIG. 5B) the blot was reacted with an anti-gBN4 rabbit serum (1:500).

Mock: non-infected CEF

M: Precision Plus Protein Standards Bio Rad #161-0374

After 20 passages, NDV-F protein ( [symbol] ) and ILTV-gB ( [symbol] ) were expressed stably in CEF infected with double recombinant HVT.

### Example 6: Induction of Anti-NDV Antibodies in Chickens Inoculated with Double Recombinant HVTs

Double recombinant HVT were inoculated subcutaneously into the back of ten one-day-old SPF chickens (LineM, NISSEIKEN) using 20 Gauge syringe. The inoculation doses are shown in Table 2. The serum was collected from the vaccinated birds, and anti-NDV antibody titers were measured by a commercial ELISA kit (IDVET, ID screen Newcastle Disease Indirect kit to diagnose Newcastle Disease). Chickens of the negative control group (non-immunized) were not administered with any vaccine.

Table 2 shows anti-NDV titer by S/P value, and the results clearly show that double recombinant HVT according to the invention induced anti-NDV antibodies as early as three weeks after inoculation, and increased anti-NDV antibodies thereafter.

TABLE 2
Induction of anti-NDV antibody in SPF chickens
inoculated with double recombinants.

| | | Average S/P value | |
|---|---|---|---|
| Group | Dose (pfu/head) | 3 weeks of age | 4 weeks of age |
| Non immunized | 0 | 0.00 | 0.00 |
| FW241 | 1350 | 0.16 | 0.46 |
| FW242 | 540 | 0.39 | 0.51 |
| FW243 | 1300 | 0.01 | 0.08 |
| FW244 | 750 | 0.36 | 0.84 |

### Example 7: Efficacy of rHVT/ND/ILT in SPF Chickens Against ILTV

The efficacy of rHVT/ND/ILT (FW241, FW242, FW243, FW244) as ILT vaccine was evaluated by a challenge with ILTV USDA strain.

Double recombinant HVT were subcutaneously inoculated into the back of one-day-old SPF chickens. At four weeks after inoculation, vaccinated chickens were challenged at intratracheally (IT) with 10³EID₅₀/bird of ILTV USDA strain. The challenged chickens were observed daily to check mortality and to detect any symptoms of infectious laryngotracheitis such as depressed, labored breathing, rales, bloody expectoration, sneezes, gasping, head flicking, stretched neck or ruffled feathers. The results at day 10 post-challenge were summarized in Table 3.

TABLE 3
Challenge experiments of rHVT/ND/ILT-vaccinated SPF chickens
with virulent ILTV US strain

| | Dose | Immu- | | | | Protection |
|---|---|---|---|---|---|---|
| Vaccine | (pfu/head) | nized | Challenged | Dead | Symptom | (%) |
| FW241 | 1350 | 10 | 9 | 0 | 3 | 67 |
| FW242 | 540 | 10 | 10 | 1 | 2 | 70 |
| FW243 | 1300 | 10 | 10 | 0 | 1 | 90 |
| FW244 | 750 | 10 | 10 | 0 | 2 | 80 |
| NICC | 0 | 0 | 10 | 3 | 5 | 20 |
| NINC | 0 | 0 | 0 | 0 | 0 | 100 |

As shown in Table 3, all of the double recombinant HVT according to the invention induced a protective immunity in chickens against the challenge with virulent ILTV.

### Example 8: Efficacy of rHVT/ND/ILT in SPF Chickens Against NDV

The efficacy of rHVT/ND/ILT (FW241, FW242, FW243, FW244) as ND vaccine was evaluated.

2,000 PFU/200 μl/bird of rHVT/ND/ILT were inoculated subcutaneously into the back of fifteen one-day-old SPF chickens (LineM, Japan Biological Laboratories) using 20 Gauge syringe. From three weeks post vaccination onward, the serum was collected from the vaccinated birds and anti-NDV antibody titer was measured by a commercial ELISA kit (IDVET, ID screen Newcastle Disease Indirect kit to diagnose Newcastle Disease).

Chickens of the negative control group were not administered with any vaccine (NI: not immunized).

At 49 days of age (48 days post vaccination), chickens of all five groups were challenged with 10³EID₅₀ of NDV-TexasGB, a strain used frequently as a challenge strain in efficacy studies, by intra-muscular injection into the femoral region. The challenged chickens were observed daily to check mortality and to detect any symptoms of Newcastle disease such as depressed, gasping, neurological symptom, and moribund. The results at day 10 post-challenge are presented in Table 4.

TABLE 4
Challenge experiments of rHVT/ND/ILT-vaccinated SPF chickens
with virulent NDV

| | | | | | | HI | ELISA |
|---|---|---|---|---|---|---|---|
| | | | | | % | (ELISA) | titer at |
| | immu- | chal- | | | Protec- | titer at | chal- |
| Vaccine | nized | lenged | Dead | Symptom | tion | hatch | lenge |
| FW241 | 15 | 15 | 0 | 0 | 100 | 0 | 0.69 |
| FW242 | 15 | 15 | 0 | 0 | 100 | | 0.77 |
| FW243 | 15 | 15 | 0 | 3 | 80 | | 0.16 |
| FW244 | 15 | 15 | 0 | 0 | 100 | | 1.26 |
| NICC | 0 | 15 | 14 | 1 | 0 | | 0.05 |
| NINC | 0 | (10) | 0 | 0 | 100 | | N/A |

As shown in Table 4, all of the double recombinant HVT according to the invention induced a protective immunity in chickens against the challenge with NDV.

1. A recombinant herpes virus of turkey (“HVT”) which comprises at least a first and a second recombinant nucleotide sequences, each recombinant nucleotide sequence encoding a polypeptide, wherein the first recombinant nucleotide sequence is inserted into the intergenic region of the viral genome located between HVT053 and HVT054, and wherein the second recombinant nucleotide sequence is inserted into an intergenic region of the viral genome located between HVT064 and HVT070.

2. The recombinant HVT of claim 1, wherein the second recombinant nucleotide sequence is inserted into the intergenic region of the viral genome located between HVT064 and HVT065.

3. The recombinant HVT of claim 1, wherein the second recombinant nucleotide sequence is inserted into the intergenic region of the viral genome located between HVT065 and HVT066.

4. The recombinant HVT of claim 1, wherein the second recombinant nucleotide sequence is inserted into the intergenic region of the viral genome located between HVT066 and HVT067.

5. The recombinant HVT of claim 1, wherein the second recombinant nucleotide sequence is inserted into the intergenic region of the viral genome located between HVT067 and HVT069.

6. The recombinant HVT of claim 1, wherein the second recombinant nucleotide sequence is inserted into the intergenic region of the viral genome located between HVT069 and HVT070.

7. The recombinant HVT of claim 1, wherein each recombinant nucleotide sequence encodes an antigen from an avian pathogen, or an immunogenic fragment thereof.

8. The recombinant HVT of claim 7, wherein each antigen is selected from surface proteins, secreted proteins and structural proteins of said avian pathogen, or an immunogenic fragment thereof.

9. The recombinant HVT of claim 7, wherein each recombinant nucleotide sequence encodes an antigen chosen among an antigen of avian paramyxovirus type 1, preferably the F protein of Newcastle disease virus (NDV) or an immunogenic fragment thereof, an antigen of Gumboro disease virus, preferably the VP2 protein of the Infectious bursal disease virus (IBDV) or an immunogenic fragment thereof, an antigen of the infectious laryngotracheitis virus (ILTV), preferably the gB protein or an immunogenic fragment thereof, an antigen of Mycoplasma gallisepticum, preferably the 40K protein or an immunogenic fragment thereof, and an antigen of the avian influenza virus, preferentially a surface protein hemagglutinin (HA) or an immunogenic fragment thereof.

10. The recombinant HVT of claim 1, wherein the first and second recombinant nucleotide sequences encode different antigens from a same or different pathogen, or immunogenic fragments thereof.

11. The recombinant HVT of claim 1, wherein each recombinant nucleotide sequence is under the control of a promoter.

12. The recombinant HVT of claim 11, wherein each promoter controlling expression of a recombinant nucleotide sequence is chosen among the chicken beta-actin (Bac) promoter, the Pec promoter, the Murine Cytomegalovirus (Mcmv) immediate-early (ie) 1 promoter, the Human Cytomegalovirus promoter (Hcmv), the Simian virus (SV) 40 promoter, and the Raus Sarcoma virus (RSV) promoter, or any fragments thereof which retain a promoter activity.

13. The recombinant HVT of claim 1, which comprise (i) inserted in the intergenic region between HVT065 and HVT066, a recombinant nucleotide sequence encoding a F protein of NDV or an immunogenic fragment thereof, preferentially under control of a Mcmv ie1 promoter and, (ii) inserted in the intergenic region between HVT053 and HVT054, a recombinant nucleotide sequence encoding a gB protein of ILTV or an immunogenic fragment thereof, preferentially under the control of a Pec promoter.

14. The recombinant HVT of claim 1, which comprises, (i) inserted in the intergenic region between HVT065 and HVT066, a recombinant nucleotide sequence encoding a F protein of NDV or an immunogenic fragment thereof, preferentially under control of a Mcmv ie1 promoter and, (ii) inserted in the intergenic region between HVT053 and HVT054, a recombinant nucleotide sequence encoding a gB protein of ILTV or an immunogenic fragment thereof, preferentially under the control of a Pec promoter.

15. The recombinant HVT of claim 1, which comprises, (i) inserted in the intergenic region between HVT065 and HVT066, a recombinant nucleotide sequence encoding a F protein of NDV or an immunogenic fragment thereof, preferentially under control of a Pec promoter and, (ii) inserted in the intergenic region between HVT053 and HVT054, a recombinant nucleotide sequence encoding a gB protein of ILTV or an immunogenic fragment thereof, preferentially under the control of a Pec promoter.

16. The recombinant HVT of claim 1, which comprises, (i) inserted in the intergenic region between HVT065 and HVT066, a recombinant nucleotide sequence encoding a gB protein of ILTV or an immunogenic fragment thereof, preferentially under control of a Mcmv ie1 promoter and, (ii) inserted in the intergenic region between HVT053 and HVT054, a recombinant nucleotide sequence encoding a F protein of NDV, or an immunogenic fragment thereof, preferentially under the control of a Pec promoter.

17. A nucleic acid comprising the genome of a recombinant HVT of claim 1.

18. A cell containing a recombinant HVT of claim 1.

19. A composition comprising a recombinant avian herpes virus of claim 1 and a suitable excipient or diluent.

20. A composition comprising a nucleic acid of claim 17 and a suitable excipient or diluent.

21. A multivalent vaccine which comprises an effective immunizing amount of a recombinant avian herpes virus of claim 1, a nucleic acid comprising the genome of the recombinant avian herpes virus and/or a cell containing the recombinant avian herpes virus.

22. A method of immunizing an avian against a pathogen comprising administering a recombinant HVT of claim 1 or a vaccine comprising the recombinant HVT to an avian.

23. A method for vaccinating an avian simultaneously against at least two pathogens comprising administering a multivalent vaccine of claim 21.

24. A vaccination kit for immunizing an avian which comprises the following components:
a. an effective amount of the multivalent vaccine of claim 21, and
b. a means for administering said vaccine to said avian.

25. A cell comprising a nucleic acid of claim 17.

26. A composition comprising a cell of claim 18 and a suitable excipient or diluent.


SEQUENCE LISTING (30 sequences)

SEQ ID NO: 1 - DNA, 1506 bp, Artificial Sequence
  Bac promoter
tgcagctcagtgcatgcacgctcattgcccatcgctatccctgcctctcctgctggcgctccccgggaggtgacttcaaggggaccgcaggaccacctcgggggtggggggagggctgcacacgcggaccccgctccccctccccaacaaagcactgtggaatcaaaaaggggggaggggggatggaggggcgcgtcacacccccgccccacaccctcacctcgaggtgagccccacgttctgcttcactctccccatctcccccccctccccacccccaattttgtatttatttattttttaattattttgtgcagcgatgggggcggggggggggggggcgcgcgccaggcggggcggggcggggccaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgaagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggggtttatatcttcccttctctgttcctccgcagccccc

SEQ ID NO: 2 - DNA, 557 bp, Artificial Sequence
  Pec promoter
tgcagagttattaatagtaatcaattacggggtcattagttcatagcccatatatggagytccgcgttacataacttacggtaaatggcccgccggctgaccgcccaacgacccccgcccattgacgtcaataatgacgtatgytcccatagtaacgccaatagggactttccattgacgtcaatgggtggagtayttacggtaaactgcccattggcagtacatcaagtgtatcatatgccaagtacgccccctattgacgtcaatgacggtaaatggatgcagtattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgt

SEQ ID NO: 3 - DNA, 572 bp, Artificial Sequence
  Mcmv ie1 promoter
ggccaataaggctgcagtactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcaggcggccgctctaga

SEQ ID NO: 4 - DNA, 20 bp, Artificial Sequence
  oligonucleotide
ggatgtccaattcgcacatg

SEQ ID NO: 5 - DNA, 19 bp, Artificial Sequence
  oligonucleotide
gctacagttacgggattca

SEQ ID NO: 6 - DNA, 30 bp, Artificial Sequence
  oligonucleotide
gggggaattcactacttttaattctcttta

SEQ ID NO: 7 - DNA, 50 bp, Artificial Sequence
  oligonucleotide
gggggccaataaggccgctagcggccgcctaacacccccgaatattagtc

SEQ ID NO: 8 - DNA, 57 bp, Artificial Sequence
  oligonucleotide
ggggcggccgctagcggccttattggcctcacgtgtagcccattgtgtgcatataac

SEQ ID NO: 9 - DNA, 34 bp, Artificial Sequence
  oligonucleotide
gggaagcttagatctgaaataacgcagttgtagg

SEQ ID NO: 10 - DNA, 30 bp, Artificial Sequence
  oligonucleotide
ggggaattcgccagatatccaaagtacagc

SEQ ID NO: 11 - DNA, 52 bp, Artificial Sequence
  oligonucleotide
gggggccaataaggccgctagcggccgccaattattttatttaataacatat

SEQ ID NO: 12 - DNA, 54 bp, Artificial Sequence
  oligonucleotide
ggggcggccgctagcggccttattggccaccagtgaacaatttgtttaatgtta

SEQ ID NO: 13 - DNA, 30 bp, Artificial Sequence
  oligonucleotide
gggaagcttgggtctgtcctagcgatataa

SEQ ID NO: 14 - DNA, 30 bp, Artificial Sequence
  oligonucleotide
gggggaattctccagattgttggatatctg

SEQ ID NO: 15 - DNA, 59 bp, Artificial Sequence
  oligonucleotide
gggggccaataaggccgctagcggccgccttattgatttataaaaacatacatgcagtg

SEQ ID NO: 16 - DNA, 57 bp, Artificial Sequence
  oligonucleotide
ggggcggccgctagcggccttattggccagtacataatttattacgtatcatttccg

SEQ ID NO: 17 - DNA, 30 bp, Artificial Sequence
  oligonucleotide
gggaagcttcctgcaagacctcatacggaa

SEQ ID NO: 18 - DNA, 30 bp, Artificial Sequence
  oligonucleotide
gggggaattcatttcttcattgcaacgacg

SEQ ID NO: 19 - DNA, 52 bp, Artificial Sequence
  oligonucleotide
gggggccaataaggccgctagcggccgcatgatcgtgctcattactgcatcg

SEQ ID NO: 20 - DNA, 54 bp, Artificial Sequence
  oligonucleotide
ggggcggccgctagcggccttattggccgggcggggcgatgacgttctatttgc

SEQ ID NO: 21 - DNA, 29 bp, Artificial Sequence
  oligonucleotide
gggaagcttaatacgcagattcttttcgg

SEQ ID NO: 22 - DNA, 30 bp, Artificial Sequence
  oligonucleotide
gggggaattctaaagaatcgtacatgagcg

SEQ ID NO: 23 - DNA, 52 bp, Artificial Sequence
  oligonucleotide
gggggccaataaggccgctagcggccgcctgatgtataagattgccgaaaag

SEQ ID NO: 24 - DNA, 53 bp, Artificial Sequence
  oligonucleotide
ggggcggccgctagcggccttattggcccgggttgcgtgaatactggtcacag

SEQ ID NO: 25 - DNA, 29 bp, Artificial Sequence
  oligonucleotide
gggaagcttacgatctggcaaaagggtcc

SEQ ID NO: 26 - DNA, 87 bp, Artificial Sequence
  oligonucleotide
ctgcaggcggccgctctagagtcgacaataaaagatctttattttcattagatctgtgtgttggttttttgtgtggccaataaggcc

SEQ ID NO: 27 - DNA, 22 bp, Artificial Sequence
  oligonucleotide
ccactgccactgtgatgataag

SEQ ID NO: 28 - DNA, 25 bp, Artificial Sequence
  oligonucleotide
gcctactatgcacattgttactcct

SEQ ID NO: 29 - DNA, 24 bp, Artificial Sequence
  oligonucleotide
ggggaagtcttccggttaagggac

SEQ ID NO: 30 - DNA, 24 bp, Artificial Sequence
  oligonucleotide
ggtgcaattcgtaagaccgatggg